(19) United States
(12) Patent Application Publication
(10) Pub. No.: US 2001/0029268 A1
(43) Pub. Date: Oct. 11, 2001

(54) Clitoral sensitizing arrangement using compound of menthol and L-arginine

(76) Inventor: Ronald J. Thompson, Ft. Thomas, KY (US)

Correspondence Address: Donald N. Halgren, 35 Central Street, Manchester, MA, 01944, US

(21) Appl. No.: 09/878,583

(22) Filed: Jun. 12, 2001

Related U.S. Application Data
(62) Division of application No. 09/469,959, filed on Dec. 21, 1999.
(63) Continuation-in-part of application No. 09/414,250, filed on Oct. 7, 1999, now Pat. No. 6,224,541.
Application No. 09/414,250 is a continuation-in-part of application No. 09/340,227, filed on Jul. 1, 1999, now Pat. No. 6,179,775.

(51) Int. Cl. A61K 31/045; A61K 31/195

(52) U.S. Cl. 514/565; 514/729

(57) ABSTRACT

The invention comprises a topical compound preparation of sensitizing cream for application to the non-keratinized epithelium of a female, comprising a preparation of L-arginine and a preparation of menthol. The preparation of L-arginine may have a concentration of 4% or less, and the preparation of menthol has a concentration of 5% or less.

This invention relates to arrangements for the stimulation of females and more particularly to topical compounds and arrangements for applying such compounds to the clitoris of females to improve their sexual sensation, and is a continuation-in-part application of U.S. patent application Ser. No. 09/414,250, filed Oct. 7, 1999, which is a continuation-in-part application of U.S. patent application Ser. No. 09/340,227, filed Jul. 1, 1999, both of which are incorporated herein by reference in their entirety.

### BACKGROUND OF THE INVENTION

1. Field of the Invention

2. Prior Art

In the human female, decreased clitoral sensitivity and responsiveness are related to normal aging, relative or absolute estrogen and testosterone deficiency (either as a consequence of medicines or aging), and by a host of vascular conditions such as diabetes and hypertension. Multiple laboratory and clinical research endeavors have been directed toward male erectile dysfunction (ED), yielding not only an understanding of the erection physiology, but also medications to treat ED, such as Viagra™. Very little research has been initiated to understand or address female physiological sexual unresponsiveness. However, since the penis and the clitoris are analogous anatomical structures, the basic cellular and physiological knowledge about male penile erections translates to functions of the clitoris.

Female clitoral dysfunction is extremely difficult to document and quantify. A number of modalities, such as Doppler blood flow, precise temperature measurements, and actual imaging measurements, have been employed to attempt to define clitoral responsiveness—all with results unsatisfactory for meaningful research. Estimates that 15 million U.S. men suffer from erectile dysfunction have been reported in the literature. A recent article in the Feb. 10, 1999 issue of the Journal of the American Medical Association suggests that female erectile dysfunction occurs probably twice the rate of male ED, therefore affecting 30 million U.S. women.

In the female anatomy, the clitoral artery is located in the middle of the clitoris, extending lengthwise from the base to the tip of the clitoris, and supplies blood to the clitoris. Two clitoral veins are located on either side of the clitoral artery, and normally drain the clitoris of the blood pumped into it from the artery. As female sexual arousal initiates, either by direct stimulation of the clitoris or by the application of one of the recently developed female arousal creams, the vessels dilate and the valves of the clitoral veins located at the base of the clitoris close. The venous blood fills two honeycomb-like chambers, the corpus cavemosa. The corpus cavemosa are normally empty of blood, but, like the clitoral artery and veins, they are positioned lengthwise from the base to the tip of the clitoris. Therefore, as the valves in the veins at the base of the clitoris close, the blood pumped into the clitoris artery distends the corpus cavemosa. This causes the clitoris to enlarge two-to-three fold, and to become erect, rigid, and highly sensitive, just as the penis in the male. In fact, the penis and the clitoris are the exact same structures. Female clitoral enlargement and rigidity and male penile erection are both accomplished by the same action: vasodilation of the vascular structures maximally distending the corpus cavemosa.

Topical application of medications to different types of Integument (skin) to effect changes in sensation are based upon that type of integument. There are two types of integument (skin): keratinized stratified squamous epithelium, and non-keratinized stratified squamous epitherlum, more commonly referred to as mucous membrane. The entire external surface of the body is covered with keratinized stratified squamous epithelium except the lips, mouth, anus, and the vagina/vulva in females. If a lotion is applied to the keratinized squamous epithelium, it is absorbed only by the top layers of the skin. Multiple transdermal medications are delivered through the keratinized skin, but the delivery system is very sophisticated. Absorption of medications is much easier if they are delivered to mucous membranes, or non-keratinized stratified squamous epithelium, especially the vulva and vaginal mucosa. Many vaginal creams and suppositories that dissolve to become creams with the moisture and heat of the vagina have been sold for over fifty years. All of the vaginal creams and suppositories are absorbed into full thickness of the non-keratinized stratified squamous epithelium that defines the vaginal mucosa. Some of the vaginal creams are systemically absorbed by the blood vessels in the basement membrane of the vaginal mucosa, while other medications are not systemically absorbed. Systemic absorption refers to the distribution of the medication throughout all tissues of the body via the blood stream. The vaginal/vulvar mucosa is a multiple layer of non-keratinized stratified squamous epithelial cells twenty-to-thirty cells in thickness from the basement membrane to the outermost cells of the mucosa.

The integument of the undercarriage of the clitoris is non-keratinized stratified squamous epithelium (mucous membrane), and topical medications are readily absorbed. The clitoral hood that covers the dorsal aspect of the clitoris is partially keratinized, and allows partial absorption of topical medications. The integument of the penis is keratinized stratified squamous epithelium. Absorption of the same medication topically applied to the penis and the clitoris concurrently will be much more readily absorbed by the clitoris because of the mucous membrane of the clitoris.

Topical menthol is a clitoral sensitizing agent and may be applied clitoral tissue and may have a number of effects. The specific effects that menthol causes are dependent upon three variables; the concentration of the menthol in the preparation, the vehicle and its evaporation properties, and most importantly, the type of tissue to which the menthol is applied. If the menthol is in a non-evaporating vehicle and applied to keratinized stratified squamous epithelium (hairy skin), a low concentration is under 1%, a midrange concentration, 1%-5%, and a high concentration, between 5% and 10%. If menthol in a non-evaporating vehicle is applied to non-keratinized stratified squamous epithelium (mucous membrane), a low concentration is less than 0.5%, a mid-range concentration between 0.5% and 2%, and a high concentration between 2% and 5%. Therefore, a 1% concentration of menthol in a non-evaporating vehicle applied to keratinized and non-keratinized epithelium would produce very different effects: the keratinized epithelium would respond with a minimal effect, while the non-keratinized epithelium would respond with a marked effect. This is because of the rapid absorption through the non-keratinized epithelium (mucous membrane) and the relatively poor absorption through the barrier-like keratinized epithelium (hairy skin).

Menthol is highly lipid-soluble and readily absorbed by a mucous membrane. A mid-range concentration of 0.5% to 2% of menthol topically applied to mucous membrane causes almost immediate smooth muscle relaxation, hyperemia, and vasodilation of blood vessels by a direct response. Menthol also causes the immediate stimulation of both thermoreceptors and nociceptors. The thermoreceptors, when stimulated, create a sensation of warmth and burning for this menthol concentration in mucous membrane (like the clitoris). The nociceptors initiate an axon reflex to release vasodilator peptides. The vasodilator peptides cause an immediate vasodilation of blood vessels with resultant blood engorgement and transudate production. The sera component of the blood that leaks out of the blood vessels to provide increased vaginal/vulvar lubrication is due to the nociceptor initiated vasodilation. The onset of all of these mechanisms occurs within thirty seconds of application of the menthol preparation, and these responses persist for fifteen-to-twenty minutes. Therefore, the topical application of a 1% menthol preparation, in a non-evaporating vehicle directly to the mucous membrane of the clitoris, causes the following physiological responses: a. warmth, tingling, and even mild burning; b. hyperemia; c. direct smooth muscle relaxation; d. indirect blood engorgement via nociceptor-mediated vasodilation by peptide release; and e. increased vulvar/vaginal lubrication.

Topical L-arginine is a clitoral sensitizing agent, produced from intracellular nitric acid (NO), which is a potent vasodilator of smooth muscles, and has been identified and confirmed by a number of research studies to be pivotally involved in penile/clitoral erections. Nitric oxide is synthesized from the substrate L-arginine in a cellular reaction catalyzed by nitric oxide synthase (NOS). Two different types of NOS have been identified: eNOS and nNOS. The eNOS is contained with the smooth muscle endothelial cells that line the vasculature of the penis/clitoris. As the available L-arginine is concerted into NO by eNOS, the blood vessels and corpus cevernosa of the penis/clitoris dilate and fill with blood. This engorgement of the penis/clitoris is the mechnism for the erection and rigidity of the aroused penis/clitoris. The NNOS is contained within the nerve cells of the penis/clitoris and converts L-arginine into NO to sensitize the penis/clitoris and cause the release of vasodilation peptide. Burnett, et al. reported in “Immunohistochemical Description of Nitric Oxide Synthase Isoforms in Human Clitoris”, Journal of Urology, July 1997, 75-79, that the vascular and neuronal alteration of the penis/clitoris that create an erection are the direct effect of the ability of the cells to convert the available L-arginine into nitric oxide through nitric oxide synthase.

Several urologic and gynecologic reports have identified a correlation between a decreased availability or activity of the NO synthase system and normal aging, testosterone deficiency, and estrogen deficiency. The studies confirm that an increase in the supply of the substrate L-arginine allows the attenuated but still functional NOS system to affect the nitric oxide medicated erection of the penis/clitoris. Therefore, the topical application of L-arginine via the NOS system causes vasodilation of the penile/clitoral veinous structures and concomitant sensitivity.

The safety of 1% of 2% menthol topically applied to the clitoris is a consideration. Two different strengths of menthol may be commercialized, such as 1% and 2% concentrations in a non-evaporating water base. The anticipated normal volume of such cream to be applied to the clitoris is 300 mg. Therefore, the maximum amount of menthol delivered is 6 mg. The Extra Pharmacopoeia, Martindale, 1989,reports menthol toxicity for humans at 28 mg./kg. In a 50 kg individual, the toxic dose could be 1400 mg., but this is estimated to be extremely low by Eccles in his “Review: Menthol and Other Cooling Compounds” in the 1994 Journal of Pharmacological Pharmacoepedia, volume 46, pages 618-630. A 6 mg. dose of menthol is well below the 1400 mg. dose calculated and gives a 100 plus fold margin of safety.

The safety of L-arginine topically applied to the clitoris is also a consideration. Two different strengths of L-arginine USP, 2% and 4%, may be marketed so as to sensitize the clitoris. The anticipated normal volume of such cream to be applied to the clitoris is 300 mg. Therefore, a maximum of 12 mg. Of L-arginine USP is available. If all 12 mg. are systemically absorbed, this is a small fraction of what has been reported in the gynecologic literature as safe. Fracchinette, et al., reported the intravenous infusion of 30 grams (30,000 milligrams) of L-arginine into 29 pregnant patients without adverse effects in the 1999 Journal of the Society of Gynecologic Investigation, volume 6,202-207.

### BRIEF SUMMARY OF THE INVENTION

Unique properties of a combination menthol/L-arginine topical clitoral sensitizing preparation provides the basis of the present invention. Sequential application first by menthol applied topically to the mucous membrane of the clitoris has an almost immediate response by affecting vasodilation and blood engorgement warmth (via the thermoreceptors). The duration of menthol's action on mucous membranes may be for fifteen-to-twenty minutes. Subsequent topical application of L-arginine utilizes its topical or systemic effect, via the NOS synthase/NO system, to create corpus cavemosa engorgement, which has a delayed onset of action from between fifteen to twenty minutes. By combining the two compounds, with simultaneous topical application of menthol and L-arginine, their effect would be immediate and prolonged because of their two different, but related, methods of action.

L-arginine absorption is promoted by the actions of menthol. Eccles reports studies by Katayama (1972), Morimoto (1993) and Yamo (1991) where the topical application of menthol, because of its local vasodilatative and lipidphillic properties, promotes the penetration of other topical drug preparation applied concurrently with menthol. These studies found rapid absorption of indomethacin, cortisone, morphine hydrochloride, and methyl salicylate when applied topically in combination with menthol. It is only rational that the absorption of L-arginine by the mucous membrane is hastened when applied in combination with menthol. This is a direct action of the lipidphillic and vasodilation properties of menthol. The action of menthol causes the L-arginine NOS/NO vasodilation to be effective within minutes of the topical application of the menthol/L-arginine combination.

A different effect is realized on the clitoris and the penis using the same menthol/L-arginine combination. This is because the integument of the clitoris is non-keratinized epithelium, a 1% menthol and 2% L-arginine preparation, topically applied, is immediately absorbed, and immediately active in sensitizing the clitoris. The penis is relatively unaffected by the application of the same strength cream because the external epithelium is keratinized squamous epithelium. The keratinized squamous epithelium, by nature of the keratin, acts as a barrier to the absorption of the preparation. Therefore, the same preparation of 1% menthol and 2% L-arginine combination sensitizes the clitoris, without sensitizing the penis. Such a combination cream may be carried in &/or on and applied by the applicators as described in the aforementioned co-pending U.S. patent application Ser. Nos. 09/414,250 and 09/349,227, each being incorporated herein by reference.

The invention thus comprises a topical compound preparation of sensitizing cream for application to the non-keratinized epithelium of a female, comprising a preparation of L-arginine, and a preparation of menthol. The preparation of L-arginine may have a concentration of 4% or less. The preparation of menthol may have a concentration of 5% or less. The preparation of L-arginine may in one embodiment have a concentration of 2% and the preparation of menthol may have a concentration of 1%.

The compound preparation may in one embodiment be enclosed within a reservoir of an applicator. The reservoir may have multiple chambers. The reservoir may have an arrangement of conduits from the reservoir to an outer clitoral-touching surface on the applicator. The invention also includes a method of sensitizing the non-keratinized epithelium of a female, comprising the steps of: applying a preparation of menthol and L-arginine to the epithelium, arranging the preparation of L-arginine in a concentration of less than 4%; and arranging the preparation of menthol in a concentration of less than 5%. The method may include the step of: placing the preparations of menthol and L-arginine in an applicator reservoir for distribution onto the epithelium, and may include the step of applying the preparations to the epithelium in a sequential manner.

### BRIEF DESCRIPTION OF THE DRAWING

The objects and advantages of the present invention will become more apparent when viewed in conjunction with the following drawing, in which:

FIG. 1 is a perspective view of an applicator for the application of the preparation of the present invention.

### DESCRIPTION OF THE PREFERRED EMBODIMENTS

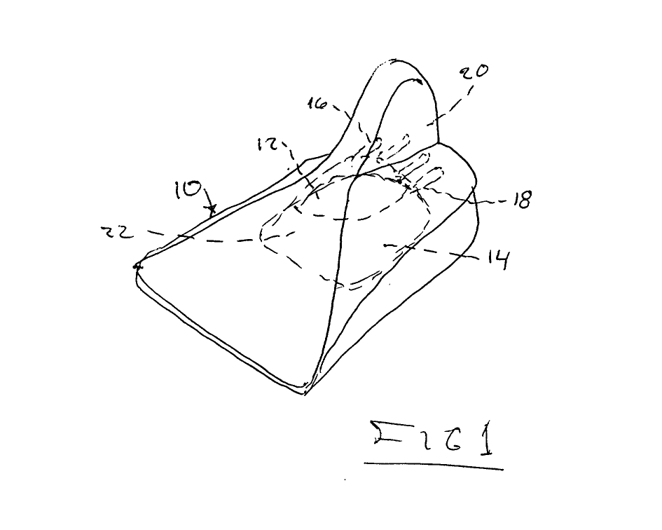

The present invention utilizes the unique properties of a combination menthol/L-arginine topical clitoral sensitizing preparation. In one embodiment, a sequential application first by menthol applied topically to the mucous membrane of the clitoris has an almost immediate response by affecting vasodilation and blood engorgement warmth (via the thermoreceptors). The duration of menthol's action on mucous membranes may be for fifteen-to-twenty minutes. Subsequent topical application of L-arginine utilizes its topical or systemic effect, via the NOS synthase/NO system, to create corpus cavernosa engorgement, which has a delayed onset of action from between fifteen to twenty minutes. Such a sequential delivery of multiple preparations may be accomplished by an applicator 10, as shown in FIG. 1. The applicator 10 may have a plurality of reservoirs 12 and 14, each with their own conduits 16 and 18 between the reservoirs 12 and 14 and a clitoral engaging surface 20. Each preparation of menthol and L-arginine may have their own rate of distribution, to provide the sequential application to the treatment situs.

In a further embodiment, a common reservoir 22 may be arranged within the applicator 10. By combining the two compounds within that common reservoir 22 as a simultaneous topical application of menthol and L-arginine together, their effect would be immediate and prolonged because of their two different, but related, methods of action. Such a range of L-arginine of less than 4% concentration and a range of menthol of less than 5% concentration is preferred in an application, either with the distribution via an applicator 10, or by other topical application.

I claim:

1. A topical compound preparation of sensitizing cream for application to the non-keratinized epithelium of a female, comprising:
a preparation of L-arginine; and
a preparation of menthol.

2. The topical compound preparation of sensitizing cream for application to the non-keratinized epithelium of a female, as recited in claim 1, wherein said preparation of L-arginine has a concentration of 4% or less.

3. The topical compound preparation of sensitizing cream for application to the non-keratinized epithelium of a female, as recited in claim 1, wherein said preparation of menthol has a concentration of 5% or less.

4. The topical compound preparation of sensitizing cream for application to the non-keratinized epithelium of a female, as recited in claim 1, wherein said preparation of L-arginine has a concentration of 2% and s aid preparation of menthol has a concentration of 1%.

5. The topical compound preparation of sensitizing cream for application to the non-keratinized epithelium of a female, as recited in claim 1, wherein said compound preparation is enclosed within a reservoir of an applicator.

6. The topical compound preparation of sensitizing cream for application to the non-keratinized epithelium of a female, as recited in claim 5, wherein said reservoir has multiple chambers.

7. The topical compound preparation of sensitizing cream for application to the non-keratinized epithelium of a female, as recited in claim 5, wherein said reservoir has an arrangement of conduits from said reservoir to an outer clitoral touching surface on said applicator.

8. A method of sensitizing the non-keratinized epithelium of a female, comprising the steps of:
applying a preparation of menthol and L-arginine to said epithelium.

9. A method of sensitizing the non-keratinized epithelium of a female, as recited in claim 8, comprising the steps of:
arranging said preparation of L-arginine in a concentration of less than 4%; and
arranging said preparation of menthol in a concentration of less than 5%.

10. The method of sensitizing the non-keratinized epithelium of a female, as recited in claim 8, comprising the step of:
placing said preparations of menthol and L-arginine in an applicator reservoir for distribution onto said epithelium.

11. The method of sensitizing the non-keratinized epithelium of a female, as recited in claim 8, comprising the steps of:
applying said preparations to the epithelium in a sequential manner.
